(19) United States
(12) Patent Application Publication
(10) Pub. No.: US 2023/0348834 A1
(43) Pub. Date: Nov. 2, 2023

(54) FIXED BED BIOREACTOR VESSEL AND METHODS OF USING THE SAME

(71) Applicant: CORNING INCORPORATED, CORNING, NY (US)

(72) Inventors: Sumitava De, Chesterfield, MO (US); Abhijit Rao, Painted Post, NY (US); Ryann Loren Russell, Drexel Hill, PA (US); Yujian Sun, Painted Post, NY (US)

(21) Appl. No.: 17/779,044

(22) PCT Filed: Oct. 30, 2020
(86) PCT No.: PCT/US2020/058079

§ 371 Date: May 23, 2022

Related U.S. Application Data
(60) Provisional application No. 62/939,957, filed on Nov. 25, 2019.
(60) Provisional application No. 62/940,384, filed on Nov. 26, 2019.

(51) Int. Cl. C12M 1/12; C12M 1/08

(52) U.S. Cl. CPC C12M 25/18; C12M 27/24

(57) ABSTRACT

A packed-bed bioreactor is provided that includes: a vessel having an interior cavity defined by an outer wall; and a center column disposed within the interior cavity. The center column includes a columnar sidewall defining an inner region within the center column, the columnar sidewall separating the inner region from an outer region within the interior cavity. The bioreactor further includes a cell culture substrate disposed in the outer region of the cavity, the cell culture substrate surrounding the center column; at least one port extending through the vessel for at least one supply and removal of media to or from the interior cavity; and a fountain head element disposed above the center column.

### CROSS REFERENCE TO RELATED APPLICATIONS

This application claims the benefit of priority under 35 U.S.C § 120 of U.S. Provisional Application Ser. No. 62/939,957 filed on Nov. 25, 2019, and U.S. Provisional Application Ser. No. 62/940,384 filed on Nov. 26, 2019, the contents of which are relied upon and incorporated herein by reference in their entireties.

### FIELD OF THE DISCLOSURE

The present disclosure generally relates to the bioprocess field and, in particular, to packed-bed bioreactors with improved flow uniformity and methods for using the bioreactor for performing a cell culture.

### BACKGROUND

In the bioprocessing industry, large scale cultivation of cells is performed for purposes of the production of hormones, enzymes, antibodies, vaccines and cell therapies. A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing.

Another example of a high-density culture system for anchorage dependent cells is a packed bed bioreactor system. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions. Another significant drawback of packed bed systems disclosed in a prior art is the inability to efficiently harvest intact viable cells at the end of culture process. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

Roller bottles have several advantages such as ease of handling, and ability to monitor cells on the attachment surface. However, from a production standpoint, the main disadvantage is the low surface area to volume ratio while the roller bottle configuration occupies a large area of manufacturing floor space. Various approaches have been used to increase the surface area available for adherent cells in a roller bottle format. Some solutions have been implemented in commercially available products, but there remains room for improvement to increase roller bottle productivity even further. Traditionally, a roller bottle is produced as a single structure by a blow-molding process. Such manufacturing simplicity enables economic viability of roller bottles in bioprocessing industry. Some roller bottle modifications to increase the available surface area for cell culturing can be achieved without changing manufacturing process, however only marginal increase of modified roller bottle surface area is obtained. Other modifications of the roller bottle design add significant complexity to manufacturing processes making it economically unviable in the bioprocessing industry. It is desirable therefore to provide roller bottle with increased surface area and bioprocessing productivity, while using the same blow-molding process for its manufacturing.

While manufacturing of viral vectors for early-phase clinical trials is possible with existing platforms, there is a need for a platform that can produce high-quality product in greater numbers in order to reach late-stage commercial manufacturing scale. In particular, there is a need for a platform and methods for compartmentalizing the packed bed while managing fluid flow of cells and nutrients through the bed, and aeration of the cell culture medium.

### SUMMARY

Disclosed herein is a packed-bed bioreactor comprising: a vessel comprising an interior cavity defined by an outer wall; a center column disposed within the interior cavity, the center column comprising a columnar sidewall defining an inner region within the center column, the columnar sidewall separating the inner region from an outer region within the interior cavity; a cell culture substrate disposed in the outer region of the cavity, the cell culture substrate surrounding the center column; at least one port extending through the vessel configured for at least one supply and removal of media to or from the interior cavity; and a fountain head element disposed above the center column. The bioreactor may further include a mixer disposed within the interior cavity and configured to circulate media through the inner region and the outer region. The fountain head element comprises a lower surface facing the inner region, the lower surface being configured to redirect media coming from the inner region toward the outer wall and downward into the outer region.

Also disclosed herein is a packed-bed bioreactor comprising: a vessel including an interior cavity defined by an outer wall, the interior cavity having a longitudinal axis between a first end and a second end of the interior cavity; a fluid inlet in the interior cavity near the first end; a fluid outlet in the interior cavity near the second end; a cell culture space disposed in the interior cavity between the fluid inlet and the fluid outlet; and a flow uniformity means disposed between the first end and the cell culture space. The interior cavity is arranged for fluid flow from the fluid inlet to the fluid outlet, a direction from the fluid inlet to the fluid outlet being a general flow direction of the interior cavity. The flow uniformity means is arranged to temporarily divert fluid flow from the general flow direction.

Additional aspects of the present disclosure will be set forth, in part, in the detailed description, figures and any claims which follow, and in part will be derived from the detailed description, or can be learned by practice of the disclosure. It is to be understood that both the foregoing general description and the following detailed description are exemplary and explanatory only and are not restrictive of the disclosure as disclosed.

### BRIEF DESCRIPTION OF THE DRAWINGS

A more complete understanding of the present disclosure may be had by reference to the following detailed description when taken in conjunction with the accompanying drawings wherein:

FIG. 1A illustrates a cross-section view of a packed-bed bioreactor according to embodiments of the present disclosure.

FIG. 1B illustrates a flow path of media through the packed-bed bioreactor of FIG. 1A, according to embodiments of the present disclosure.

FIG. 2 shows a bottom view of the bioreactor of FIGS. 1A and 1B, according to embodiments of the present disclosure.

FIG. 3 illustrates a cross-section view of a packed-bed bioreactor according to embodiments of the present disclosure.

FIG. 4A illustrates a perspective view of a center column of a bioreactor vessel, according to embodiments of the present disclosure.

FIG. 4B is a cross-section view of the center column of FIG. 4A, according to embodiments of the present.

FIG. 5A illustrates a bioreactor center column with a fountain head element, according to embodiments of the present disclosure.

FIG. 5B illustrates a close-up view of the fountain head element of FIG. 5A, according to embodiments of the present disclosure.

FIG. 5C illustrates a bottom view of the fountain head element of FIGS. 5A and 5B, according to embodiments of the present disclosure.

FIG. 5D illustrates the center column and fountain head element of FIG. 5A with a fountain head spray of media, according to embodiments of the present disclosure.

FIG. 6 shows a schematic view of a bioreactor, according to one or more embodiments;

FIG. 7A illustrates a bioreactor having a fluid inlet for direct flow to a cell culture space;

FIG. 7B illustrates a bioreactor having a fluid inlet with a diverter, according to one or more embodiments;

FIG. 7C illustrates a bioreactor having an inverted fluid inlet, according to one or more embodiments;

FIGS. 8A-8C show computational fluid dynamics (CFD) modeling results of the fluid flow velocity vectors in the bioreactors of FIGS. 7A-7C, respectively;

FIGS. 9A-9C show computational fluid dynamics (CFD) modeling results of the contours of velocity magnitude of fluid flow in the bioreactors of FIGS. 7A-7C, respectively;

FIGS. 10A-10C show computational fluid dynamics (CFD) modeling results of the contours of velocity magnitude of fluid flow across a horizontal plane of the bioreactors of FIGS. 7A-7C, respectively;

FIG. 11 is a schematic illustrating a bioprocessing system incorporating a packed-bed bioreactor according to embodiments of the present disclosure.

FIG. 12 shows an operation for controlling a perfusion flow rate of a cell culture system, according to one or more embodiments.

### DETAILED DESCRIPTION

Disclosed herein is a new packed-bed bioreactor which is configured for performing a cell culture. According to one or more embodiments, the packed-bed bioreactor includes: (i) a vessel having an interior cavity defined by an outer wall; (ii) a center column disposed within the interior cavity, the center column having a columnar sidewall defining an inner region within the center column that separates the inner region from an outer region within the interior cavity; (iii) a cell culture substrate disposed in the outer region of the cavity and surrounding the center column; (iv) at least one port extending through the vessel, wherein the at least one port is configured for at least one supply and removal of media to or from the interior cavity; and (v) a fountain head element disposed above the center column.

As an aspect of some embodiments, the bioreactor also includes a mixer. In embodiments, the mixer includes an impeller device having an impeller and a shaft, where the impeller and the shaft are disposed within the inner region. As an aspect of some embodiments, the vessel can be provided in multiple pieces that can be assembled into a sealed system containing the cell culture substrate. For example, the vessel may include top and bottom portions that are screwed together or otherwise joined via methods known in the art (e.g., welding, adhesive, etc.). In some embodiments, the vessel has a main body with an opening, and at least one lid attachable to the vessel to cover the opening. The mixer can be attached to the lid, or attached to a rod extending from or passing through the lid.

The at least one port can include a fresh media port and/or a spent media port. The fresh media port and the spent media port can be provided in separate or distinct ports of the vessel. Alternatively, the same port of the vessel can act as both the fresh media port and the spent media port. In such an embodiment, media and other components (e.g., cells, nutrients, etc.) can be supplied through the port at the start of the cell culture and as needed throughout the cell culture process, and the media and other components can also be removed from the vessel through the same port. For example, as an aspect of some embodiments, the cultured cells are harvested (e.g., removed) from the vessel through the port.

Also disclosed herein is a method for using the packed-bed bioreactor to perform a cell culture. The method includes the steps of: (i) providing the packed-bed bioreactor; (ii) adding cells to the outer region; (iii) introducing fresh media through the at least one port into the inner region; and (iv) operating the mixer device to provide agitation to the contents of the inner region and to propel media through the inner region to the fountain head element, where the media is redirected toward the outer wall of the vessel and down into the cell culture substrate in the outer region. The mixer enables transportation of media, nutrients, cells, and/or cell products or secreted material (e.g., recombinant protein, antibody, virus particles, DNA, RNA, sugars, lipids, biodiesel, inorganic particles, butanol, metabolic byproducts) to flow through the cell culture substrate. The method may further include removing the spent media and/or the cell byproducts or secreted material through the at least one port from the outer region. According to an aspect of some embodiments, the media is redirected by the fountain head element in an environment of oxygen so that the media is oxygenated before proceeding down through the cell culture substrate.

Various embodiments of the present disclosure will be discussed with reference to the figures, which illustrate various aspects of packed-bed bioreactors and related methods of using the bioreactors according to non-limiting embodiments of the present disclosure. The following description is intended to provide an enabling description of the bioreactor and the various aspects of the bioreactor will be specifically discussed in detail throughout the disclosure with reference to the non-limiting embodiments, these embodiments are interchangeable with one another within the context of the disclosure.

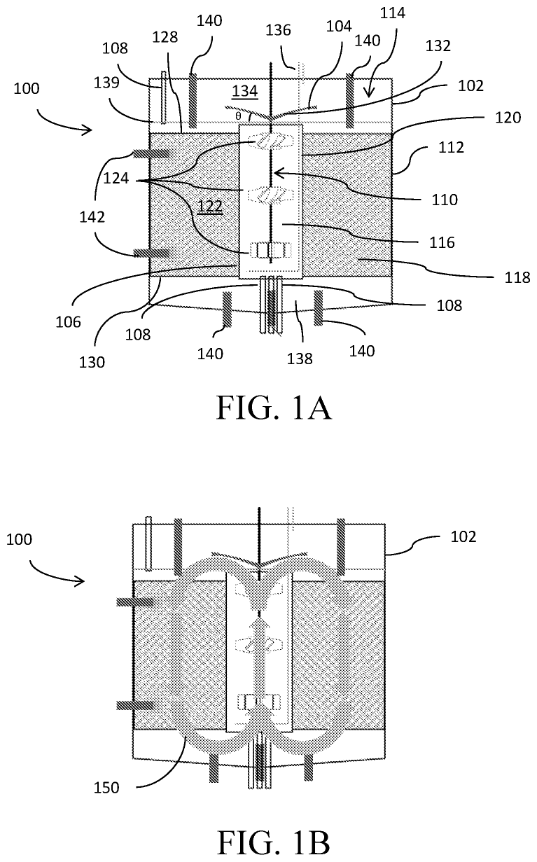

Referring to FIGS. 1A and 1B, there is a schematic illustrating the basic components of a packed-bed bioreactor 100 in accordance with an embodiment of the present disclosure. As shown, the bioreactor 100 includes a vessel 102, a fountain head element 104, a center column 106, one or more ports 108, and a mixer 110. The vessel 102 has an outer wall 112 defining a cavity 114 within. The center column 106 is disposed within the cavity 114 in a manner to divide the cavity 114 into an inner region 116 and an outer region 118 that are separated by a sidewall 120 of the center column 106. The inner region 116 is designed to transport cell culture media from the one or more ports 108 to the outer region 118 (as shown by the arrows 150 in FIG. 1B). The outer region 118 is designed to accommodate a cell culture substrate 122 used for culturing cells.

According to embodiments of this disclosure, the cell culture substrate 122 is provided within the outer region 118 of the cavity 114 and includes one or more porous bodies having a solid material on or within which cells can be cultured. According to some embodiments, the bioreactor 100 is used for culturing adherent cells on the surface(s) of the cell culture substrate 122. In addition, an aspect of embodiments allows for the culture of non-adherent or loosely adherent cells within interstitial spaces of the cell culture substrate 122. The cell culture substrate 122 may be provided in a packed-bed configuration in which a number of pieces of substrate material are held together within the outer region 118. Alternatively, the cell culture substrate 122 may take the form of a monolithic porous material within the outer region 118.

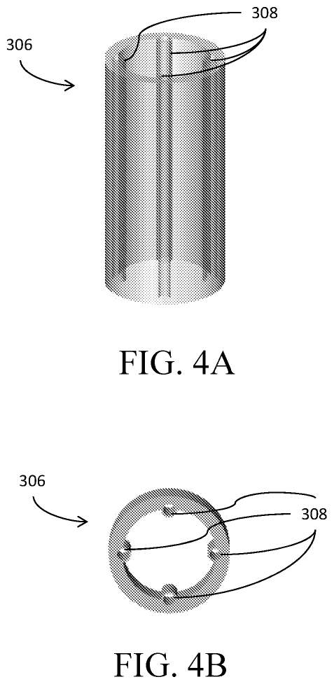

The mixer 110 can include one or more impellers 124. The impeller can be designed to optimize one or both of mixing of components within the cavity 114 and propulsion of those components through the center column 106. One or both of the inner region and the outer region can contain motional stirrers or impellers, according to some embodiments. The impeller design can be tailored to maximize transport along the longitudinal axis of the center column 106 while yielding the proper cell agitation. For example, an impeller 124 that has blades that run parallel to the longitudinal axis of the center column 106 may facilitate mixing of components of the cell culture media and break up of sparge bubbles to aid in driving gasses into solution (i.e., a Rushton style impeller). Additionally, or alternatively, the impeller 124 may have blades that are angled or curved to push media through the center column 106. The mechanical motion of pushing media through the center column 106 and into the cell culture substrate 122 without damaging cells or other components is the desired effect. The mixer 110 includes the impeller 124 and a shaft 132. The impeller 124 and the shaft 132 are both disposed within the inner region 116. In this example, multiple impellers 124 are attached at various points along the length of the shaft 132, while the top end of the shaft 132 is rotatably attached to and extends downward from the top of the vessel 102. In an alternative embodiment, as shown in FIG. 3, an impeller 224 can disposed on the bottom of the cavity 214 and can be rotated by a magnetic stir plate (not shown) located under the vessel 202. Alternatively, the mixer 210 may have a boat style top down driven impeller, or the mixer may be a levitating stir element, a magnetic stir element, or a paddle-like stirring element. In embodiments, any suitable stirring devices may be used.

The fountain head element 104 is provided at one end of the center column 106 in the path of media transported through the center column 106. The fountain head element 104 has a bottom surface 132 shaped to redirected media impinging thereon toward the outer wall 112 of the cavity 114, and ultimately downward into the cell culture substrate 122. To redirect the impinging fluid, the bottom surface 132 of the fountain head element 104 may be provided at an angle θ relative to the direction of flow of the fluid through the center column 106 (i.e., at an angle relative to the longitudinal axis of the center column 106). The angle θ may be greater than 0° and less than or equal to 90°. In some preferred embodiments, the angle θ is between about 1° and 70°, including at least about 2°, at least about 3°, at least about 4°, at least about 5°, at least about 6°, at least about 7°, at least about 8°, at least about 9°, at least about 10°, at least about 12°, at least about 14°, at least about 15°, at least about 20°, at least about 25°, up to about 65°, up to about 60°, up to about 55°, up to about 50°, up to about 45°, up to about 40°, up to about 35°, up to about 30°, up to about 25°, up to about 20°, up to about 15°, or up to about 10°. In some embodiments, the angle θ varies with the distance from the longitudinal axis of the center column 106. For example, the bottom surface 132 may be curved with a constant or variable curvature.

The bottom surface 132 of the fountain head element 104 is shaped so that incident fluid is redirected in a cascading fashion outward and downward toward and through the outer region 118 and/or cell culture substrate 122. As an aspect of some preferred embodiments, the fountain head element 104 redirects the impinging media in a thin film to maximize the gas-media interface between the media and the surrounding gaseous environment of a headspace 134 in the top portion of the cavity 114. The headspace 134 is supplied with one or more components desired to be exchanged into the cell culture media. The one or more components include oxygen and other nutrients for the cell culture. Thus, the thin film action of the fountain head element 104 creates a zone where oxygenation can occur separate any sparged zone that may be present elsewhere in the cavity 114. For example, according to one or more embodiments, a sparge element 136 is provided to supply oxygen or other fluid to the cavity 114. As shown in FIGS. 1A and 1B, the sparge element 136 can enter through a top of the vessel 102 and be positioned to release a fluid (e.g., oxygen) in the inner region 116 or a lower region 138 of the vessel 102. In this way, any one of or all of the headspace 134, the lower region 138 in the bottom of the cavity 114, and the inner region 116 can be enriched with air or oxygen. The oxygenation can thus be controlled to maintain oxygen setpoint as cells grow and their oxygen demand increases. The bottom surface of the lower region 138 may also be sloped to aid in retrieval or drainage of components from the cavity, according to some embodiments.

The size of the center column 106, the speed of media ejecting from the top of the center column 106, and geometry of the fountain head element 104 will dictate the spread of the fountain effect. These factors can be adjusted (e.g., defining a minimum agitation speed, and thus flow rate out of the central column, to maintain the fountain effect) in such a way that maximizes oxygenation but minimizes any risk of excessive foam formation (especially in high serum containing media of, e.g., 10% fetal bovine serum). The fountain head can be controlled to impinge upon the outer wall 112 of the vessel 102, rather than impinging directly onto the media fill line 139, so that the likelihood of entraining air into the medium, which could lead to foam, can be reduced. Nonetheless, the bioreactor 100 may include an inlet line for addition of antifoam reagents typical in bioreactors (e.g., Dow Corning® Medical Antifoam C).

As the cell culture medium progresses through the cell culture substrate 122, it is depleted due to cell metabolism. When the medium exits the cell culture substrate 122, it enters the lower region 138 of the bioreactor. In the lower region 138, sensors 140 (e.g., probes, single use patches, RAMAN, etc.) read environmental conditions (e.g., pH, CO₂, DO, temperature, fluid flow, shear stress, cell density) to allow for monitoring and control of the bioreactor 100 via, for example, a PID feedback loop to the system. As an example, should the differential of dissolved oxygen (DO) be too great as medium exits the bed, the center column 106 can increase flow rate via increased rotational speed thus increasing flow through the cell culture substrate 122 and decreasing the DO differential due to the shorter medium residence time within the packed-bed. In addition, the one or more ports may provide inlets to allow liquid feeds (e.g., caustic, glucose, media, bolus addition) to immediately feed into a high turbulence zone within the center column 106 such that the fluids mix well into the medium prior to coming into contact with the fixed bed. In addition, one or more probes 142 (e.g., biomass probes, Raman probes) may be supplied in or near the cell culture substrate 122. As shown by the arrows 150 in FIG. 1B, the medium that has traveled through the cell culture substrate 122 can be recirculated by the mixer 110 through the center column 106 to repeat the cycle. As the medium is recirculated, it can be adjusted using additions from the one or more ports 108 and/or based on measurements from the sensors 140, as described above.

As shown in FIGS. 1A and 1B, a plurality of ports 108 are provided through the top and bottom of the vessel 102. The ports are configured to supply or remove one or more of cell culture media, cells, cell by-products, cell culture nutrients, oxygen, caustic, nitrogen, and other common additives or byproducts of cell culture known in the art. As shown in FIGS. 1A and 1B, a plurality of ports 108 can be provided. Alternatively, the vessel 102 may be provided with a single port through which all desired components can be added and/or removed from the cavity 114. The one or more ports 108 can be supplied by one or more tubes from components external to the cavity 114.

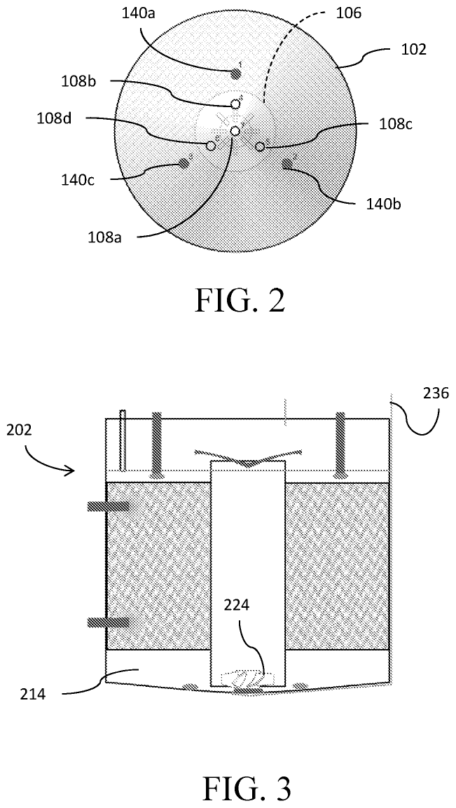

FIG. 2 shows a bottom view of the vessel 102 showing a plurality of ports 108a-108d for fluid input or removal, and a plurality of sensor ports 140a-c for one or more sensors. As shown, the plurality of ports 108a-108d are provided below the center column 106 for easy feeding of medium and component into the flow path of the center column 106. The plurality of sensor ports 140a-c is provided below the outer region 118 to measure the depleted medium coming from the above cell culture substrate 122. The arrangement of ports in FIG. 2 is provided for example only, and embodiments of this disclosure are not limited to the number or arrangement of ports shown. In addition, as shown in FIG. 1B, additional sensors 140d and 140e, and an inlet 108e can be provided through the top of the vessel 102 for supplying medium, nutrients, oxygen, etc. to the medium in the headspace 134 of the cavity 114.

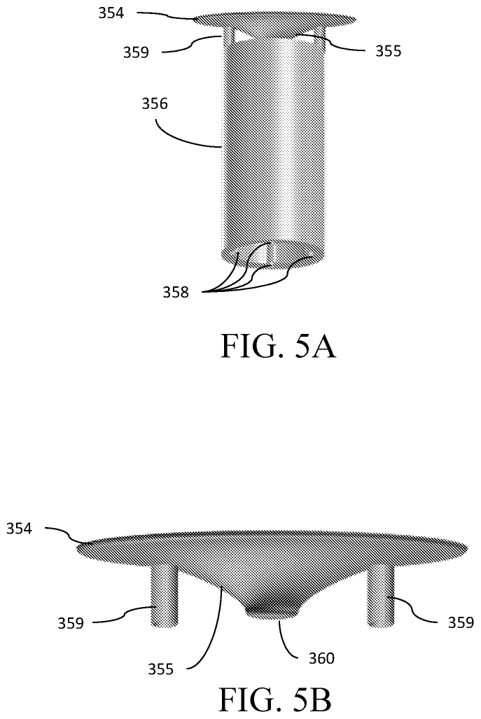

According to one or more embodiments, as shown in FIGS. 4A and 4B, the vessel 102 can include a center column 306 that is an extruded piece with integrated feed lines 308 such that feeds come if from the top of the reactor 100 but still terminate at the bottom of the center column 306 (in place of center column 106 in FIG. 1A) for the same advantaged mixing. Sparge gasses could come down these integrated feed lines 308 and have a sparge element that plugs into the line as a part of system assembly. This configuration would could allow for simpler deployment of the reactor consumable into a chassis or holder (bioreactor instrument) due to the need of having feed tubing fluidly connected to the system. Sparge may also occur via a sparge line that penetrates from the bottom of the vessel, as shown in FIG. 3.

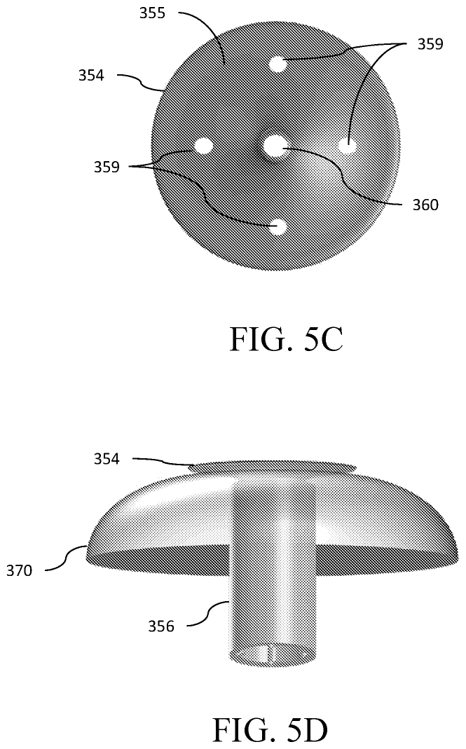

FIG. 5A illustrates a fountain head element 354 positioned on a center column 356 with integrated feed lines 408, according to one or more embodiments. The center column 356 has a similar structure to the center column 356 of FIGS. 4A and 4B. FIG. 5B is a cross-section view of the fountain head element 354 showing feed lines 359 to feed the integrated feed lines 358 of the center column 356, and the curved bottom surface 355 used to redirect media impinging on the surface 355 from the center column. As shown in FIG. 5C, these feed lines 359 can be spaced circumferentially around the fountain head element 354 so that they can be connected to each of the integrated feed lines 358 of the center column 356. In addition, the fountain head element 354 includes a center opening 360 to accommodate a mixer arm, such as that shown in FIGS. 1A and 1B. FIG. 5D shows the resulting fountain head of media 370 formed by the redirection of media by the fountain head element 354. The shape of the fountain head of media 370 can be adjust by changing the shape of the lower surface 355 and adjusting the flow rate of media through the center column 356. It may be desirable to adjust the shape of the fountain head of media 370 so that foaming is minimized when the media re-enters the remaining media of the vessel at a fill line.

The cell culture substrate 122 is porous to allow perfusion of cells, media, nutrients, and cell by-products through the substrate within the inner region 118 and allow spent media with cell secreted material (e.g., recombinant protein, antibody, virus particles, DNA, RNA, sugars, lipids, biodiesel, inorganic particles, butanol, metabolic byproducts) to pass through into the outer region 118. Further details of the cell culture substrate according to embodiments are provided below. The cell culture substrate 122 can be held within the outer region 118 of the cavity 114 by a wide-variety of methods including, for example, injection over-molding, adhesives, laminate membranes, spot welding, laser sintering, and ultrasonic welding. According to some embodiments, the cell culture substrate 122 is held within the outer region 118 between a top plate 128 and a bottom plate 130, which are porous members that provide sufficient rigidity to hold the cell culture substrate 122 in place while allowing media, cells, and other fluids to pass through them.

The vessel 102 can be plastic, glass, ceramic or stainless steel. According to some embodiments, all or part of the vessel 102 may be made of a transparent material or may include one or more transparent windows in the outer wall 112 to allow for inspection of the interior of the vessel 102 via the human eye or any of a number of sensors, probes, cameras, or monitoring units. For example, according to an aspect of some embodiments, an optical camera or Raman spectroscopy probe can be used to monitor the cell culture progress within the cavity 114.

In embodiments, an optional lid may be removably attached to the vessel, or may be permanently attached to the vessel. In embodiments, then lid is integral to the vessel, allowing the perfusion bioreactor, once assembled, to be a closed, integral device. Or, alternatively, the lid when removable allows the perfusion bioreactor to be disassembled by the user and the contents to be accessed by the user.

As described above, a bioreactor system for cell culture is provided that enables an efficient fluid flow path, including mixing and mass exchange (e.g., oxygenation) of a cell culture medium. Additional aspects of some embodiments provide for flow uniformity through the packed bed cell culture substrate of the bioreactor. Improved flow uniformity through the cell culture substrate can lead to more uniform cell seeding throughout the packed bed, more uniform distribution of cell culture medium and cell nutrients, more uniform proliferation of cells during culture, decreased risk of damage to cells due to variations in flow velocity and sheer stress, and more uniform and efficient cell harvest throughout the packed bed.

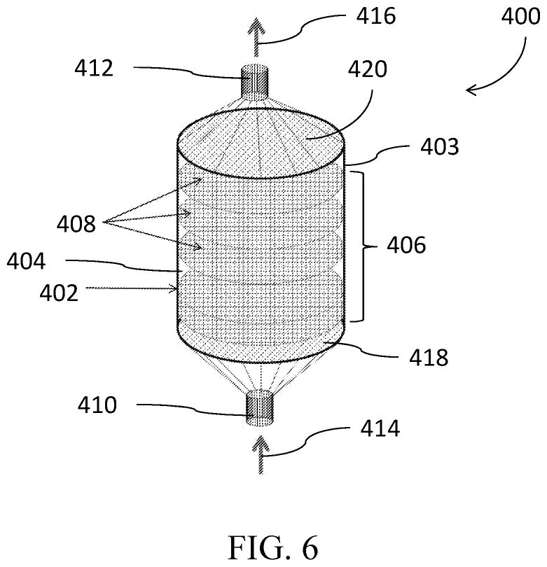

One of the challenges to achieving flow uniformity of the cell culture medium in a bioreactor vessel relates to the difficulty in establishing uniform flow at the fluid inlet of the vessel. For example, FIG. 6 shows a simplified cell culture system 400 that includes a bioreactor vessel 402 with a wall 403 defining a cell culture chamber 404 in the interior of the bioreactor vessel 402, according to some embodiments. Within the cell culture chamber 404 is a cell culture matrix 406. In the example shown, the cell culture matrix is made from a stack of substrate layers 408. In particular, the substrate layers 408 are porous discs that are stacked with the first or second side of each disc facing a first or second side of an disc. For example, the substrate layers 408 may be discs of woven polymer mesh made from a plurality of interwoven fibers defining porous interstices, as described herein. The bioreactor vessel 402 has an inlet 410 at one end for the input of media, cells, and/or nutrients into the culture chamber 404, as indicated by the arrow 414. The bioreactor vessel 402 further includes an outlet 412 at the opposite end for removing media, cells, or cell products from the culture chamber 404, as indicated by arrow 416. By allowing stacking of substrate layers in this way, the system can be easily scaled up without negative impacts on cell attachment and proliferation, due to the defined structure and efficient fluid flow through the stacked substrates. The bioreactor vessel 402 may also include a perforated plate 418 between the inlet 410 and the cell culture matrix 406. The perforated plate 418 can provide structural support to the packed-bed matrix above and/or can be used to alter a flow characteristic of fluid entering the cell culture matrix 406 from the inlet 410. Similarly, a porous member 420 may be positioned between the cell culture matrix 406 and the outlet 412. As illustrated below, the flow uniformity of fluid from the inlet 410 into the culture chamber 404 can be approved according to aspects of embodiments of the present disclosure.

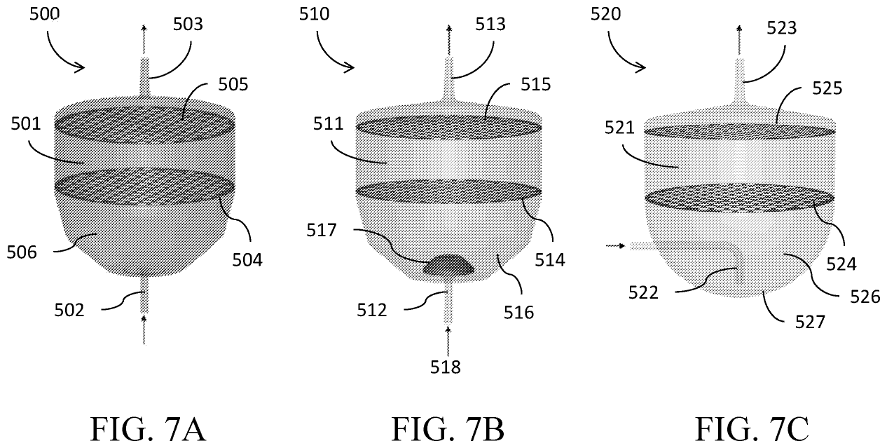

FIGS. 7A-7C illustrate three example bioreactor vessels that will be used to show the improved fluid flow provided by some embodiments. For clarity, cell culture substrates are omitted from FIGS. 7A-7C. FIG. 7A shows a bioreactor vessel 500 similar to the one in FIG. 6. Specifically, the bioreactor vessel has an internal cavity with a cell culture space 501 between an inlet 502 and an outlet 503 of the bioreactor vessel 500. The cell culture space 501 is bounded by a sidewall of the bioreactor vessel 500, a lower perforated plate 504, and an upper perforated plate 505. The inlet 502 supplies fluid directly into an inlet space 506 and can flow directly to the lower perforated plate 504.

In FIG. 7B, a bioreactor vessel 510 is provided with a similar structure to that of bioreactor vessel 500. Specifically, bioreactor vessel 510 includes an internal cavity with a cell culture space 511 between an inlet 512 and an outlet 513 of the bioreactor vessel 510. The cell culture space 511 is bounded by a sidewall of the bioreactor vessel 510, a lower perforated plate 514, and an upper perforated plate 515. However, in contrast to the bioreactor vessel 500, the bioreactor vessel 510 includes a diverter 517 in the inlet space 516 positioned between the inlet 512 and the lower perforated plate 514. The diverter 517 interferes with the inflow of fluid from inlet 512 so that fluid is dispersed or diverted from the flow direction 518 of the inlet 512. In this way, the incoming fluid is dispersed such that, by the time the fluid reaches the lower perforated plate 514, a more uniform flow across the width of the cell culture space 511 is achieved.

In FIG. 7C, a bioreactor vessel 520 is provided that includes an internal cavity with a cell culture space 521 between an inlet 522 and an outlet 523 of the bioreactor vessel 520. The cell culture space 521 is bounded by a sidewall of the bioreactor vessel 520, a lower perforated plate 524, and an upper perforated plate 525. However, in contrast to the bioreactor vessel 500, the inlet 522 of bioreactor vessel 520 is pointed toward the bottom 527 of the bioreactor vessel 520 such that fluid from the inlet 522 enters the vessel 520 traveling away from the cell culture space 521, whereas the inlet 502 of bioreactor vessel 500 is pointed toward the cell culture space 501 such that fluid enters the bioreactor vessel 500 traveling toward the cell culture space 501. By having an inlet 522 that diverts the incoming fluid away from the cell culture space, the incoming fluid will be dispersed, and flow will be more uniform when the fluid reaches the lower perforated plate 514. In addition, the bioreactor vessel 520 can have a bottom 527 that is shaped to aid in this dispersion and/or redirection of the incoming fluid. For example, in FIG. 7C, the interior surface of the bottom 527 of the vessel 520 is curved in a concave manner with respect to the interior of the vessel 520. Thus, fluid exiting the inlet 522 may be directed outward and upward from the point of entering the vessel 520 at inlet 522. In this way, by the time the fluid reaches the lower perforated plate 524, a more uniform flow across the width of the cell culture space 521 is achieved compared to that of bioreactor vessel 500.

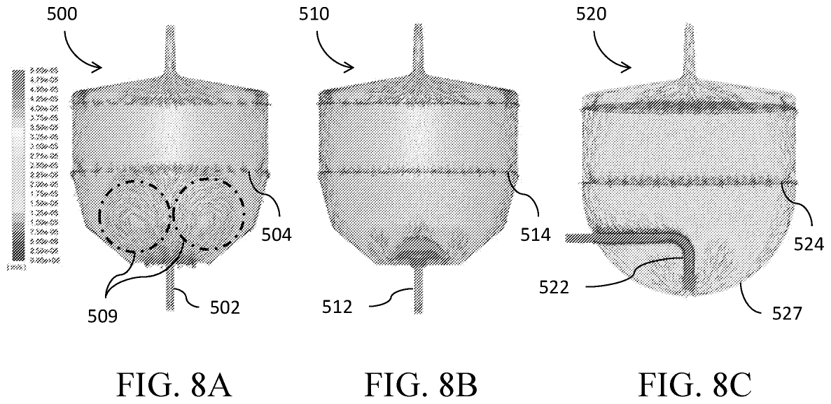

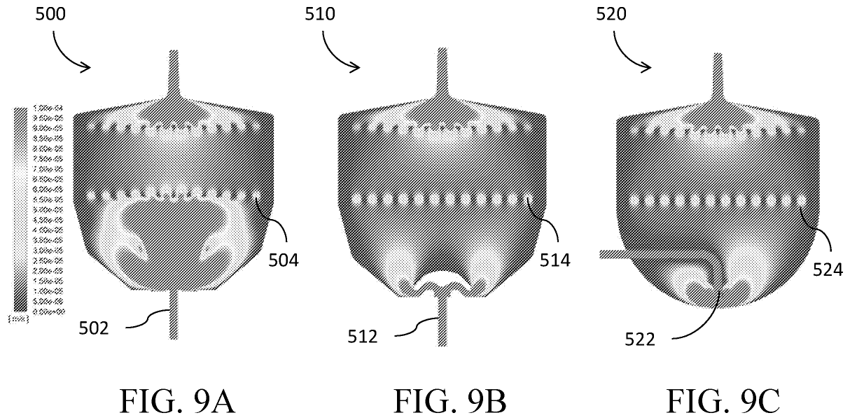

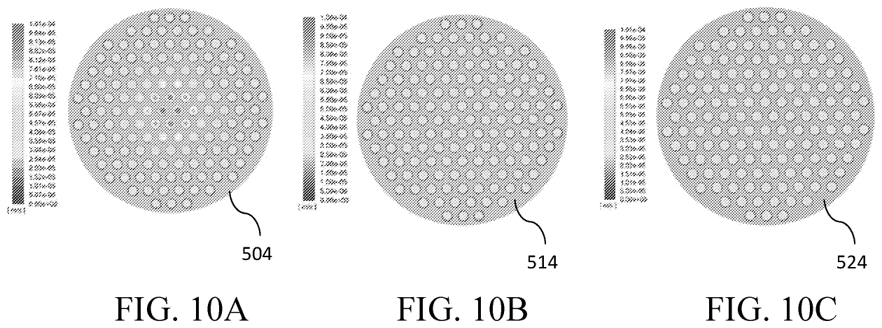

The improvements in flow uniformity in FIGS. 7B and 7C have been demonstrated by computational fluid dynamics (CFD) modeling, the results of which are shown in FIGS. 8A-10C. A volumetric flow rate of 2.1 ml/min was specified at the inlet in all models. The CFD modeling results for the three embodiments of FIGS. 7A-7C are shown side-by-side in each of FIGS. 8(A-C), 9(A-C), and 10(A-C) so that the differences in fluid flow can be readily appreciated. Specifically, FIGS. 8A, 9A, and 10A show results for the embodiment shown in FIG. 7A, FIGS. 8B, 9B, and 10B shows results for the embodiment shown in FIG. 7B, and FIGS. 8C, 9C, and 10C show results for the embodiment shown in FIG. 7C. In particular, the results in FIGS. 8A-8C show the modeled fluid flow in terms of color-coded velocity vectors of the modeled fluid in each embodiment of FIGS. 7A-7C; the results in FIGS. 9A-9C show the modeled fluid flow in terms of the contours of velocity magnitude of fluid flow in each embodiment of FIGS. 7A-7C; and the results in FIGS. 10A-10C show the contours of velocity magnitude across a plane near the lower perforated plates 504, 514, 524, respectively. In the color-coding of flow velocity in FIGS. 8A-10C, red indicates the upper end of the flow velocity range and blue indicates the lower end of the range.

As shown in FIGS. 8A, 9A, and 10A, the modeled fluid in bioreactor vessel 500 tends to take the path of least resistance resulting in high flow rates proximal to the center or longitudinal axis of the bioreactor vessel. The result is an uneven flow rate across the width of the vessel 500, including zones of high velocity fluid flow near the middle of the lower perforated plate 504. The high velocities near the longitudinal axis of bioreactor vessel 500 also create recirculation zones 509 in the inlet space 506. This non-uniform flow across the width of the bioreactor 500 could negatively impact cell culture, as cells may be distributed non-uniformly, nutrient distribution may be non-uniform, and shear stresses experimented by the cells during culture and/or harvest will be non-uniform.

In contrast, FIGS. 8B, 9B, and 10B show that the bioreactor vessel 510 has much more uniform flow due to the diverter 517. For example, the high velocity flow vectors quickly dissipate in the inlet space 516 near the diverter 517. By the time the lower perforated plate 514 is reached, the flow rates are very uniform across most of the lower perforated plate 514. Similarly, FIGS. 8C, 9C, and 10C show that the bioreactor vessel 520 has improved flow uniformity by the time fluid reaches the lower perforated plate 525 due to the downward pointing inlet 522 and concave bottom surface of the bioreactor vessel 520.

A coefficient of variation of flow velocity magnitude (CV_{f}) was calculated based on the modeling results shown in FIGS. 8A-10C. The CV_{f} is a ratio of the standard deviation of the flow velocity magnitude across a horizontal plane of the bioreactor vessel to the mean value of the flow velocity magnitude across the horizontal plane. A lower value for CV_{f} indicates a more uniform flow across the horizontal plane of the bioreactor. The embodiment of FIGS. 7A, 8A, 9A, and 10A has a CV_{f} of 0.39 across the plane of the lower perforated plate. The embodiment of FIGS. 7B, 8B, 9B, and 10B has a CV_{f} of 0.27 across the plane of the lower perforated plate. The embodiment of FIGS. 7C, 8C, 9C, and 10C has a CV_{f} of 0.33 across the plane of the lower perforated plate. Thus, the CV_{f} for the bioreactor vessels 510 and 520 is lower than the CV_{f} of bioreactor vessel 500, indicating more uniform fluid flow across the lower perforated plate.

The improved inlet configurations disclosed herein are shown and described with respect to illustrative embodiments of bioreactor vessels. However, the improved inlet configurations are not limited to the specific bioreactor vessels discussed here, and can be incorporated into a number of vessel designs. For example, the inlet designs of the present disclosure may be used in the bioreactor vessels described in U.S. patent application Ser. Nos. 16/781,685, 16/781,723, 17/039,035, and 17/039,218 and U.S. Provisional Patent Application No. 62/939,957.

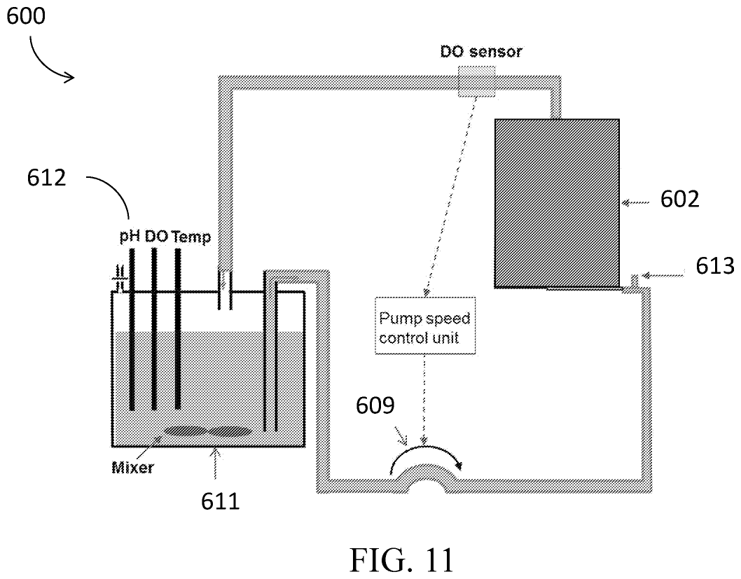

FIG. 11 shows a bioreactor 602 as described herein incorporated into a bioprocessing system 600, according to one or more embodiments. The system 600 includes a media conditioning vessel 611 for proper maintenance of cell culture media parameters such as pH, temperature, and oxygenation level, for example. Automatically controlled pump 609 is used to perfuse media through the bioreactor 602. Bioreactor inlet 613 is equipped with additional 3-way port to facilitate cell inoculation or collection of harvested cells. The system 600 may include in-line sensors, as well as the sensors in the media conditioning vessel 611.

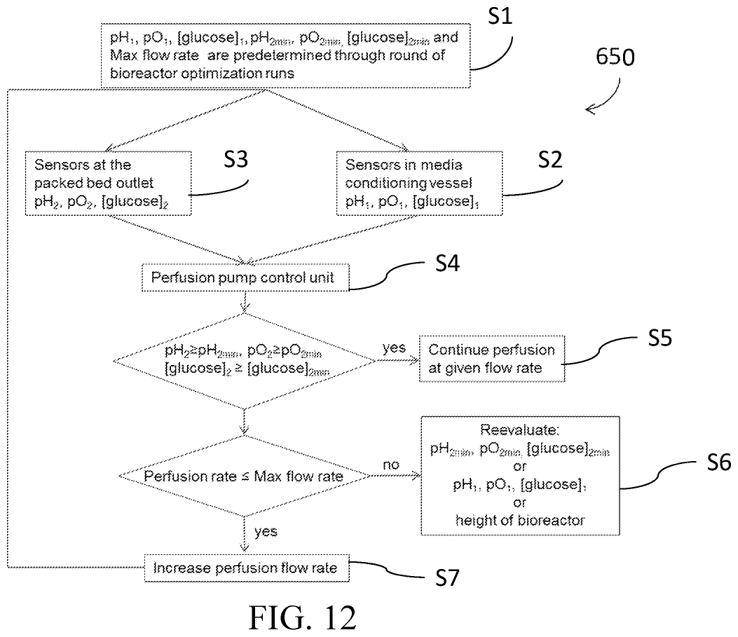

As described above, the bioreactor according to embodiments of this disclosure can include one or more ports and sensors for monitoring and adjusting of medium and cell culture environment within the vessel. However, according to some embodiments, cell culture media sensing and conditioning can be performed in a second vessel that is external to the bioreactor. For example, FIG. 11 demonstrates the schematics of bioreactor vessel 602 that is connected to main external components comprised of a media conditioning vessel, a pump allowing the flow of media into the bioreactor and external dissolved oxygen sensor that support required process conditions for successful bioprocess. Cell culture media is conditioned in media conditioning vessel 611, where proper pH, temperature and dissolved oxygen levels are maintained. Subsequently media is perfused through the bioreactor by pump 609. The flow rate of pump 609 is integrated into a feedback loop which is automatically adjusts to maintain minimal predefined level of dissolved oxygen in media exiting the bioreactor. All transfection reagents, nutrients and additional media supplements required by given bioprocess can be introduced into bulk media and spent media can removed via the media conditioning vessel 611. At the end of the process, media can be drained from bioreactor and refilled with cells harvesting solution 622 (FIG. 12). After incubating the packed bed in harvesting solution for predefined time that is sufficient for cells to detach from the substrate cells are harvested by reverse flow through applying air pressure at bioreactor outlet to achieve flow rate in a range of 70 ml/cm² (cross sectional packed bed area)/min. Cells are harvested at the bioreactor 3-way port 613. Cells can also be lysed directly in the bioreactor and lysate solution containing AVV particles can be collected through 3-way port 613.

The media conditioning vessel 604 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 604 also contains an impeller for media mixing. All media parameters measured by sensors listed above can be controlled by a media conditioning control unit 618 in communication with the media conditioning vessel 604, and capable of measuring and/or adjusting the conditions of the cell culture media 606 to the desired levels.

The media from the media 606 conditioning vessel 604 is delivered to the bioreactor 602 via an inlet, which may also include an injection port for cell inoculum to seed and begin culturing of cells. The bioreactor vessel 602 may also include on or more outlets through which the cell culture media exits the vessel 602. In addition, cells or cell products may be output through the outlet. To analyze the contents of the outflow from the bioreactor 602, one or more sensors 612 may be provided in the line. In some embodiments, the system 600 includes a flow control unit for controlling the flow into the bioreactor 602. For example, the flow control unit may receive a signal from the one or more sensors 612 and, based on the signal, adjust the flow into the bioreactor 602 by sending a signal to a pump (e.g., peristaltic pump) upstream of the inlet 608 to the bioreactor 602. Thus, based on one or a combination of factors measured by the sensors 612, the pump can control the flow into the bioreactor 602 to obtain the desired cell culturing conditions.

The media perfusion rate is controlled by the signal processing unit that collects and compares sensors signals from media conditioning vessel 604 and sensors located at the packed bed bioreactor outlet. Because of the pack flow nature of media perfusion through the packed bed bioreactor 602, nutrients, pH and oxygen gradients are developed along the packed bed. The perfusion flow rate of the bioreactor can be automatically controlled by the flow control unit operably connected to the peristaltic pump, according to the flow chart in FIG. 12.

FIG. 12 shows an example of a method 650 for controlling the flow of a perfusion bioreactor system, such as the system 600 of FIG. 11. According to the method 650, certain parameters of the system 600 are predetermined at step S1 through bioreactor optimization runs. From these optimization runs, the values of pH₁, pO₁, [glucose]₁, pH₂, pO₂, [glucose]₂, and maximum flow rate can be determined. The values for pH₁, pO₁, and [glucose]¹ are measured within the cell culture chamber of the bioreactor 602 at step S2, and pH₂, pO₂, and [glucose]₂ are measured by sensors 612 in the media conditioning vessel 604 at step S3 (or in the bioreactor according to embodiment discussed herein). Based on these values at S2 and S3, a perfusion pump control unit makes determinations at S4 to maintain or adjust the perfusion flow rate. For example, a perfusion flow rate of the cell culture media to the cell culture chamber may be continued at a present rate if at least one of pH₂≥pH₂ₘᵢₙ, pO₂≥pO₂ₘᵢₙ, and [glucose]₂≥[glucose]₂ₘᵢₙ (S5). If the current flow rate is less than or equal to a predetermined max flow rate of the cell culture system, the perfusion flow rate is increased (S7). Further, if the current flow rate is not less than or equal to the predetermined max flow rate of the cell culture system, a controller of the cell culture system can reevaluate at least one of: (1) pH₂ₘᵢₙ, pO₂ₘᵢₙ, and [glucose]₂ₘᵢₙ; (2) pH₁, pO₁, and [glucose]₁, and (3) a height of the bioreactor vessel (S6).

One or more embodiments of this disclosure provide a method for using the bioreactors described herein to perform a cell culture in a packed-bed substrate. First, the bioreactor is provided, wherein the bioreactor comprises: (i) a vessel having an interior cavity defined by an outer wall; (ii) a center column disposed within the interior cavity, the center column having a columnar sidewall defining an inner region within the center column that separates the inner region from an outer region within the interior cavity; (iii) a cell culture substrate disposed in the outer region of the cavity and surrounding the center column; (iv) at least one port extending through the vessel, wherein the at least one port is configured for at least one supply and removal of media to or from the interior cavity; (v) a fountain head element disposed above the center column; and (vi) a mixer disposed coaxially with the center column and arranged to propel medium up through the center column. Next, cells are added to the outer region 118. Fresh media is then introduced through the one of the ports 108 into the lower region 138. The mixer 110 is operated to rotate one or more impellers 124 to mix and/or transport medium up through the center column 106 to the fountain head element 104, at which point the medium is redirected in a fountain head cascade or thin sheet/film of fluid to the cell culture substrate in the outer region 118. The medium then flows down through the cell culture substrate. Transportation of spent media and cell secreted material (e.g., recombinant protein, antibody, virus particles, DNA, RNA, sugars, lipids, biodiesel, inorganic particles, butanol, metabolic byproducts) can be performed through the porous substrate 122 into the lower region 138. Optionally, the spent media and the cell secreted material are removed through the one or more ports 108.

One or more embodiments of this disclosure also includes a method of using a bioreactor vessel, where the method provides more uniform fluid flow across the packed-bed. The method includes inserting fluid into the vessel through an inlet and obstructing or dispersing the inserted fluid within the vessel before the fluid reaches the packed bed. According to an aspect of some embodiments, this is achieved by using a flow diverter place between the inlet and the cell culture space. The diverter is positioned so that fluid entering the interior space of the vessel from the inlet impinges up the diverter and is diverted in a direction that is substantially non-parallel to a direction from the inlet to the cell culture substrate. The diverter may have a flat or curved surface facing the inlet to redirect the fluid. For example, the curved surface may concave to divert the fluid radially and/or downward (i.e., away from the cell culture substrate). According to another aspect of some embodiments, fluid flow may be made more uniform by inserting fluid into the vessel so that the fluid exits the fluid inlet in a direction other than a direction toward the cell culture substrate. For example, the fluid inlet may be pointed down toward the bottom of the vessel and away from the cell culture substrate, so that the fluid enters the vessel going in a direction opposite to the direction of the cell culture substrate. In addition, a bottom interior surface of the vessel may be shaped to redirect the fluid radially and/or upward toward the cell culture substrate.

Embodiments of the present disclosure include bioreactors and cell culture substrates used therein, including substrates that are cell growth matrices and/or packed-bed systems for anchorage dependent cells that enable easy and effective scale-up to any practical production scale for cells or cell derived products (e.g., proteins, antibodies, viral particles). In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, mechanically stable, non-degradable woven meshes can be used to support adherent cell production. Uniform cell seeding of such a matrix is achievable, as well as efficient harvesting of cells or other products of the bioreactor. In addition, the embodiments of this disclosure support cell culturing to achieve confluent monolayer or multilayer of adherent cells on disclosed matrix, and can avoid formation of 3D cellular aggregates with limited nutrient diffusion and increased metabolite concentrations. The structurally defined matrix of one or more embodiments enables complete cell recovery and consistent cell harvesting from the packed bed of bioreactor. In another embodiment of the present disclosure, a method of cell culturing is provided using bioreactors with the matrix for bioprocessing production of therapeutic proteins, antibodies, viral vaccines, or viral vectors.

In one or more embodiments, the cell culture matrix supports attachment and proliferation of anchorage dependent cells in a high volumetric density format. The matrix can be assembled and used in a bioreactor system, such as a perfused back bed bioreactor as described herein, and provide uniform cell distribution during the inoculation step, while preventing formation of large and/or uncontrollable cell aggregates inside the matrix or packed bed. Thus, the matrix eliminates diffusional limitations during operation of the bioreactor. In addition, the matrix enables easy and efficient cell harvest from the bioreactor.

The matrix can be formed with a substrate material that of a thin, sheet-like construction having first and second sides separated by a relatively small thickness. In other words, the thickness of the sheet-like substrate is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways to obtain uniform cell seeding, uniform media perfusion, and efficient cell harvest.

The cell culture substrate can be a woven mesh layer made of a first plurality of fibers running in a first direction and a second plurality of fibers running in a second direction. The woven fibers of the substrate form a plurality of openings. The size and shape of the openings can vary based on the type of weave (e.g., number, shape and size of filaments; angle between intersecting filaments, etc.). An opening can be defined by a certain width or diameter. A woven mesh may be considered, on a macro-scale, a two-dimensional sheet or layer. However, a close inspection of a woven mesh reveals a three-dimensional structure due to the rising and falling of intersecting fibers of the mesh. Thus, a thickness of the woven mesh may be thicker than the thickness of a single fiber.

The woven mesh can be comprised of monofilament or multifilament polymer fibers. In one or more embodiments, a monofilament fiber may have a diameter in a range of about 50 μm to about 1000 μm. On a microscale level, due to the scale of the fiber compared to the cells (e.g., the fiber diameters being larger than the cells), the surface of monofilament fiber is presented as regular 2D surface for adherent cells to attach and proliferate. Such fibers are woven into a mesh that has a defined pattern and a certain amount of structural rigidity. Fibers can be woven into a mesh with openings ranging from about 100 μm×100 μm to about 1000 μm×1000 μm. These ranges of the filament diameters and opening diameters are examples of some embodiments, but are not intended to limit the possible feature sizes of the mesh according to all embodiments.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different structure patterns or weaves, including, for example knitted, warp-knitted, or woven (plain weave, twilled weave, dutch weave, five needle weave).

The surface chemistry of the mesh filaments may need to be modified to provide desired cell adhesion properties. Such modifications can be made through the chemical treatment of the polymer material of mesh or grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry.

The woven mesh substrate may be provided in a number of discs with a center hole configured to surround the center column of the bioreactor described herein. A plurality of such discs can be stacked in the outer region of the bioreactor to form the packed bed.

According to some embodiments, the cell culture substrate is a dissolvable foam scaffold comprising an ionotropically crosslinked polygalacturonic acid compound selected from at least one of: pectic acid; partially esterified pectic acid, partially amidated pectic acid and salts thereof; and at least one first water-soluble polymer having surface activity.

Cell culture substrates of embodiments of the present disclosure may be, for example, those disclosed in U.S. patent application Ser. Nos. 16/781,685, 16/781,723, 17/039,035, and 17/039,218 and U.S. Provisional Patent Application No. 62/939,957, the contents of which are incorporated herein by reference in their entirety.

Embodiments of this disclosure can achieve viral vector platforms of a practical size that can produce viral genomes on the scale of about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

The following aspects of embodiments of this disclosure provide illustrative examples of the embodiments described herein.

Aspect 1 is directed to a packed-bed bioreactor comprising: a vessel comprising an interior cavity defined by an outer wall; a center column disposed within the interior cavity, the center column comprising a columnar sidewall defining an inner region within the center column, the columnar sidewall separating the inner region from an outer region within the interior cavity; a cell culture substrate disposed in the outer region of the cavity, the cell culture substrate surrounding the center column; at least one port extending through the vessel configured for at least one supply and removal of media to or from the interior cavity; and a fountain head element disposed above the center column.

Aspect 2 is directed to the packed-bed bioreactor of Aspect 1, further comprising a mixer disposed within the interior cavity and configured to circulate media through the inner region and the outer region.

Aspect 3 is directed to the packed-bed bioreactor of Aspect 2, wherein the mixer is an impeller disposed substantially coaxially with the center column and is configured to force media up through the inner region of the center column and into the fountain head element.

Aspect 4 is directed to the packed-bed bioreactor of Aspect 3, wherein the impeller is attached to one end of a shaft, and wherein another end of the shaft is rotatably attached to and extends downward from a top of the vessel.

Aspect 5 is directed to the packed-bed bioreactor of any of Aspects 2-4, wherein the mixer is configured to create a turbulence zone within the center column to mix one or more fluids.

Aspect 6 is directed to the packed-bed bioreactor of Aspect 5, wherein the one or more fluids comprise at least one of caustic, cell culture media, oxygen, nitrogen, and cell culture nutrients.

Aspect 7 is directed to the packed-bed bioreactor of Aspect 2, wherein the mixer device further comprises a magnetic stir plate located external to the vessel, and wherein the magnetic stir plate is configured to rotate the mixer.

Aspect 8 is directed to the packed-bed bioreactor of any of Aspects 1-7, wherein the fountain head element comprises a lower surface facing the inner region, the lower surface being configured to redirect media coming from the inner region toward the outer wall and downward into the outer region.

Aspect 9 is directed to the packed-bed bioreactor of Aspect 8, wherein the fountain head element is configured to redirect the media in a thin film outward from the fountain head element.

Aspect 10 is directed to the packed-bed bioreactor of Aspect 8 or 9, wherein the lower surface of the fountain head element is curved.

Aspect 11 is directed to the packed-bed bioreactor of Aspect 10, wherein a lowest point of the lower surface is proximal to a longitudinal axis of the center column and the lower surface rises in a curved arc as the lower surface extends outward from the longitudinal axis of the center column toward the outer wall.

Aspect 12 is directed to the packed-bed bioreactor of any of Aspects 8-11, wherein the media that is redirected by the fountain head element is exposed to oxygen before entering the outer region.

Aspect 13 is directed to the packed-bed bioreactor of any of Aspects 8-12, wherein the fountain head element is configured to redirect the media at an angle and velocity such that the media contacts the outer wall before contacting the cell culture substrate in the outer region.

Aspect 14 is directed to the packed-bed bioreactor of Aspect 13, wherein an angle between the outer wall and the redirected media contacting the outer wall is less than 90°.

Aspect 15 is directed to the packed-bed bioreactor of any of Aspects 1-14, further comprising a controller configured to control at least one of media supply, oxygen supply, caustic supply, media temperature, cell nutrient supply, media flow rate, and mixer speed.

Aspect 16 is directed to the packed-bed bioreactor of any of Aspects 1-15, further comprising a capture zone disposed below the cell culture substrate, the capture zone being configured to capture spent media that has passed through the cell culture substrate.

Aspect 17 is directed to the packed-bed bioreactor of Aspect 16, wherein the capture zone comprises one or more sensors configured to measure one or more conditions in the spent media.

Aspect 18 is directed to the packed-bed bioreactor of Aspect 17, wherein the one or more conditions comprise pH, dissolved oxygen, temperature, composition, analyte levels, and spectral characteristics.

Aspect 19 is directed to the packed-bed bioreactor of Aspect 17 or 18, wherein the controller is configured to adjust a property of the packed-bed bioreactor based on a signal from the one or more sensors.

Aspect 20 is directed to the packed-bed bioreactor of any of Aspects 1-19, wherein the at least one port comprises a media inlet, a media outlet, an oxygen inlet, a caustic inlet, a cell nutrient inlet, a pH sensor, an oxygen sensor, a temperature probe, an analyte sensor, a sparge line, and a biomass sensor.

Aspect 21 is directed to the packed-bed bioreactor of any of Aspects 1-20, wherein the cell culture substrate comprises a porous material.

Aspect 22 is directed to the packed-bed bioreactor of Aspect 21, wherein the cell culture substrate comprises at least one of polystyrene, polyethylene terephthalate, polycarbonate, polyvinylpyrrolidone, polybutadiene, polyvinylchloride, polyethylene oxide, polypyrroles, and polypropylene oxide.

Aspect 23 is directed to the packed-bed bioreactor of Aspect 21 or Aspect 22, wherein the cell culture substrate comprises at least one of a molded polymer lattice, a 3D-printed polymer lattice sheet, and a woven mesh sheet.

Aspect 24 is directed to the packed-bed bioreactor of any of Aspects 21-23, wherein the cell culture substrate comprises the woven mesh comprising one or more fibers.

Aspect 25 is directed to the packed-bed bioreactor of Aspect 24, wherein the one or more fibers have a fiber diameter from about 50 μm to about 1000 μm, from about 50 μm to about 600 μm, from about 50 μm to about 400 μm, from about 100 μm to about 325 μm, or from about 150 μm to about 275 μm.

Aspect 26 is directed to the packed-bed bioreactor of Aspect 24 or Aspect 25, wherein the woven mesh comprises a plurality of openings interstitial to the one or more fiber, the plurality of openings having a diameter of from about 100 μm to about 1000 μm, from about 200 μm to about 900 μm, or from about 225 μm to about 800 μm.

Aspect 27 is directed to the packed-bed bioreactor of Aspect 21, wherein the cell culture substrate is a dissolvable foam scaffold.

Aspect 28 is directed to the packed-bed bioreactor of Aspect 27, wherein the dissolvable foam scaffold comprises: an ionotropically crosslinked polygalacturonic acid compound selected from at least one of: pectic acid; partially esterified pectic acid, partially amidated pectic acid and salts thereof; and at least one first water-soluble polymer having surface activity.

Aspect 29 is directed to the packed-bed bioreactor of Aspect 27 or Aspect 28, wherein the dissolvable foam scaffold comprises an adhesion polymer coating.

Aspect 30 is directed to the packed-bed bioreactor of Aspect 29, wherein the adhesion polymer coating comprises peptides.

Aspect 31 is directed to the packed-bed bioreactor of Aspect 30, wherein the adhesion polymer coating comprises peptides selected from the group consisting of BSP, vitronectin, fibronectin, laminin, Type I collagen, Type IV collagen, denatured collagen and mixtures thereof.

Aspect 32 is directed to the packed-bed bioreactor of Aspect 29, wherein the adhesion polymer coating comprises Synthemax® II-SC.

Aspect 33 is directed to a packed-bed bioreactor comprising: a vessel comprising an interior cavity defined by an outer wall, the interior cavity comprising a longitudinal axis between a first end and a second end of the interior cavity; a fluid inlet in the interior cavity near the first end; a fluid outlet in the interior cavity near the second end; a cell culture space disposed in the interior cavity between the fluid inlet and the fluid outlet; and a flow uniformity means disposed between the first end and the cell culture space, wherein the interior cavity is configured for fluid flow from the fluid inlet to the fluid outlet, a direction from the fluid inlet to the fluid outlet being a general flow direction of the interior cavity, and wherein the flow uniformity means is configured to temporarily divert fluid flow from the general flow direction. Aspect 33 can further include any of the features in any one of Aspects 1-32.

Aspect 34 is directed to the packed-bed bioreactor of Aspect 33, further comprising a first flow plate disposed between the fluid inlet and the cell culture space, the first flow plate being configured to allow fluid to flow through the first flow plate.

Aspect 35 is directed to the packed-bed bioreactor of Aspect 33 or Aspect 34, further comprising a second flow plate disposed between the cell culture space and the fluid outlet, the second flow plate being configured to allow fluid to flow through the second flow plate.

Aspect 36 is directed to the packed-bed bioreactor of any one of Aspects 33-35, wherein the flow uniformity means is configured to provide substantially uniform flow across a width of the cell culture space.

Aspect 37 is directed to the packed-bed bioreactor of any one of Aspects 33-36, wherein the flow uniformity means comprises a diverter cap disposed between the fluid inlet and the cell culture space, the diverter cap comprising a diversion surface facing the fluid inlet and configured to divert fluid impinging on the diversion surface from the general flow direction.

Aspect 38 is directed to the packed-bed bioreactor of Aspect 37, wherein the diversion surface comprises a width that is wider than a width of the fluid inlet.

Aspect 39 is directed to the packed-bed bioreactor of Aspect 37 or Aspect 38, wherein the diversion surface is flat, convex, or concave.

Aspect 40 is directed to the packed-bed bioreactor of any one of Aspects 33-36, wherein the flow uniformity means comprises the fluid inlet facing the first end of the interior cavity such that fluid entering the interior cavity from the fluid inlet flow toward the first end immediately upon exiting the fluid inlet.

Aspect 41 is directed to the packed-bed bioreactor of Aspect 40, wherein the fluid uniformity means further comprises a concave surface facing the fluid inlet, the concave surface being configured to redirect fluid from the fluid inlet radially or toward the cell culture space.

Aspect 42 is directed to the packed-bed bioreactor of any one of Aspects 33-41, wherein a coefficient of variation of flow velocity magnitude (CV_{f}) of fluid in the cell culture space is about 0.40 or less, about 0.39 or less, about 0.38 or less, about 0.37 or less, about 0.36 or less, about 0.35 or less, about 0.34 or less, about 0.33 or less, about 0.32 or less, about 0.31 or less, about 0.30 or less, about 0.29 or less, about 0.28 or less, about 0.27 or less, about 0.26 or less, 0.25 or less, about 0.24 or less, about 0.23 or less, about 0.22 or less, about 0.21 or less, or about 0.21 or less.

Aspect 43 is directed to the packed-bed bioreactor of any one of Aspects 33-42, further comprising a cell culture substrate disposed in the cell culture space.

Aspect 44 is directed to the packed-bed bioreactor of Aspect 43, wherein the cell culture substrate comprises a porous material.

Aspect 45 is directed to the pack-bed bioreactor of Aspect 43 or Aspect 42, wherein the cell culture substrate comprises at least one of polystyrene, polyethylene terephthalate, polycarbonate, polyvinylpyrrolidone, polybutadiene, polyvinylchloride, polyethylene oxide, polypyrroles, and polypropylene oxide.

Aspect 46 is directed to the packed-bed bioreactor of any one of Aspects 43-45, wherein the cell culture substrate comprises at least one of a molded polymer lattice, a 3D-printed polymer lattice sheet, and a woven mesh sheet.

Aspect 47 is directed to the packed-bed bioreactor of any one of Aspects 43-46, wherein the cell culture substrate comprises the woven mesh comprising one or more fibers.

Aspect 48 is directed to the packed-bed bioreactor of Aspect 47, wherein the one or more fibers have a fiber diameter from about 50 μm to about 1000 μm, from about 50 μm to about 600 μm, from about 50 μm to about 400 μm, from about 100 μm to about 325 μm, or from about 150 μm to about 275 μm.

Aspect 49 is directed to the packed-bed bioreactor of Aspect 47 or Aspect 48, wherein the woven mesh comprises a plurality of openings interstitial to the one or more fiber, the plurality of openings having a diameter of from about 100 μm to about 1000 μm, from about 200 μm to about 900 μm, or from about 225 μm to about 800 μm.

Aspect 50 is directed to the packed-bed bioreactor of Aspect 43, wherein the cell culture substrate is a dissolvable foam scaffold.

Aspect 51 is directed to the packed-bed bioreactor of Aspect 50, wherein the dissolvable foam scaffold comprises: an ionotropically crosslinked polygalacturonic acid compound selected from at least one of: pectic acid; partially esterified pectic acid, partially amidated pectic acid and salts thereof; and at least one first water-soluble polymer having surface activity.

Aspect 52 is directed to the packed-bed bioreactor of Aspect 50 or Aspect 51, wherein the dissolvable foam scaffold comprises an adhesion polymer coating.

Aspect 53 is directed to the packed-bed bioreactor of Aspect 52, wherein the adhesion polymer coating comprises peptides.

Aspect 54 is directed to the packed-bed bioreactor of Aspect 53, wherein the adhesion polymer coating comprises peptides selected from the group consisting of BSP, vitronectin, fibronectin, laminin, Type I collagen, Type IV collagen, denatured collagen and mixtures thereof.

Aspect 55 is directed to the packed-bed bioreactor of Aspect 52, wherein the adhesion polymer coating comprises Synthemax® II-SC.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been illustrated in the accompanying Drawings and described in the foregoing Detailed Description, it should be understood that the disclosure is not limited to the disclosed embodiments, but is capable of numerous rearrangements, modifications and substitutions without departing from the disclosure as set forth and defined by the following claims.

1. A packed-bed bioreactor comprising:
a vessel comprising an interior cavity defined by an outer wall;
a center column disposed within the interior cavity, the center column comprising a columnar sidewall defining an inner region within the center column, the columnar sidewall separating the inner region from an outer region within the interior cavity;
a cell culture substrate disposed in the outer region of the cavity, the cell culture substrate surrounding the center column;
at least one port extending through the vessel configured for at least one supply and removal of media to or from the interior cavity; and
a fountain head element disposed above the center column.

2. The packed-bed bioreactor of claim 1, further comprising a mixer disposed within the interior cavity and configured to circulate media through the inner region and the outer region.

3. The packed-bed bioreactor of claim 2, wherein the mixer is an impeller disposed substantially coaxially with the center column and is configured to force media up through the inner region of the center column and into the fountain head element.

4. (canceled)

5. The packed-bed bioreactor of claim 2, wherein the mixer is configured to create a turbulence zone within the center column to mix one or more fluids.

6. The packed-bed bioreactor of claim 5, wherein the one or more fluids comprise at least one of caustic, cell culture media, oxygen, nitrogen, and cell culture nutrients.

7. (canceled)

8. The packed-bed bioreactor of claim 1, wherein the fountain head element comprises a lower surface facing the inner region, the lower surface being configured to redirect media coming from the inner region toward the outer wall and downward into the outer region.

9. The packed-bed bioreactor of claim 8, wherein the fountain head element is configured to redirect the media in a thin film outward from the fountain head element.

10. The packed-bed bioreactor of claim 8, wherein the lower surface of the fountain head element is curved.

11. The packed-bed bioreactor of claim 10, wherein a lowest point of the lower surface is proximal to a longitudinal axis of the center column and the lower surface rises in a curved arc as the lower surface extends outward from the longitudinal axis of the center column toward the outer wall.

12. The packed-bed bioreactor of claim 8, wherein the media that is redirected by the fountain head element is exposed to oxygen before entering the outer region.

13. The packed-bed bioreactor of claim 8, wherein the fountain head element is configured to redirect the media at an angle and velocity such that the media contacts the outer wall before contacting the cell culture substrate in the outer region.

14. The packed-bed bioreactor of claim 13, wherein an angle between the outer wall and the redirected media contacting the outer wall is less than 90°.

15. The packed-bed bioreactor of claim 1, further comprising a controller configured to control at least one of media supply, oxygen supply, caustic supply, media temperature, cell nutrient supply, media flow rate, and mixer speed.

16. The pack-bed bioreactor of claim 1, further comprising a capture zone disposed below the cell culture substrate, the capture zone being configured to capture spent media that has passed through the cell culture substrate.

17. The packed-bed bioreactor of claim 16, wherein the capture zone comprises one or more sensors configured to measure one or more of pH, dissolved oxygen, temperature, composition, analyte levels, and spectral characteristics in the spent media.

18. (canceled)

19. The packed-bed bioreactor of claim 17, wherein the controller is configured to adjust a property of the packed-bed bioreactor based on a signal from the one or more sensors.

20. The packed-bed bioreactor of claim 1, wherein the at least one port comprises a media inlet, a media outlet, an oxygen inlet, a caustic inlet, a cell nutrient inlet, a pH sensor, an oxygen sensor, a temperature probe, an analyte sensor, a sparge line, and a biomass sensor.

21. The packed-bed bioreactor of claim 1, wherein the cell culture substrate comprises a porous material.

22. (canceled)

23. The packed-bed bioreactor of claim 21, wherein the cell culture substrate comprises at least one of a molded polymer lattice, a 3D-printed polymer lattice sheet, and a woven mesh sheet.

24-26. (canceled)

27. The packed-bed bioreactor of claim 21, wherein the cell culture substrate is a dissolvable foam scaffold.

28-32. (canceled)
